(19) United States
(12) Patent Application Publication
(10) Pub. No.: US 2012/0165870 A1
(43) Pub. Date: Jun. 28, 2012

(54) PLATE-AND-SCREW IMPLANT ASSEMBLY FOR BONE FIXATION

(75) Inventor: Philippe Prestigiacomo, Lacanau-Ocean (FR)

(73) Assignee: CREASPINE, Pessac (FR)

(21) Appl. No.: 13/393,976

(22) PCT Filed: Aug. 27, 2010
(86) PCT No.: PCT/FR10/51793

§ 371 Date: Mar. 2, 2012

(30) Foreign Application Priority Data
Sep. 2, 2009 (FR) 09/04153

(51) Int. Cl. A61B 17/70

(52) U.S. Cl. 606/246

(57) ABSTRACT

The invention relates to an implant assembly for bone fixation, and particularly for stabilizing the vertebral column, including a plate (P) provided with at least two openings and at least two anchoring means (V) that can be positioned in said openings and be attached to the bone portions to be stabilized, and blocking means for preventing said anchoring means (V) from being accidentally released from said openings. Said blocking means include a shoulder (7) arranged on each anchoring means (V), and a non-return lip (L) which is arranged inside each opening, is integral with said plate (P), and can be deformed so as to be cleared by said shoulder (7) during the positioning of the anchoring means (V), and then prevent the extraction of the anchoring means (V) by forming an abutment against said shoulder (7).

### TECHNICAL FIELD

The present invention relates to an implant assembly for bone fixation, and in particular to stabilize the vertebral column.

### BRIEF DISCUSSION OF RELATED ART

It is known, in bone surgery and in particular in surgery of the spinal column, to use plates provided with openings intended to receive anchoring elements, in particular screws that can be screwed into the bone parts one wishes to immobilize or stabilize relative to one another.

This type of application is in particular found in the field of vertebral column surgery, and more particularly in the cervical vertebrae zone.

In that zone, the stabilization plates are often implanted through the anterior route, i.e. through the front of the patient's body, such that the screws are also screwed from the front of the body.

If, for various reasons (degeneration of the vertebral bodies, poor application of the implant placement protocol, etc.), the screws are made to leave the openings of the plates, they can injure the adjacent organs, and in particular the patient's esophagus in the case of an anterior cervical implant.

It is therefore crucial to provide means making it possible to prevent the screws from spontaneously leaving the openings of the stabilization plate.

Many systems are found in the prior art that aim to resolve this problem: in Anglo-Saxon terminology, reference is commonly made to “anti-blackout” systems, i.e. systems making it possible to prevent the screws from coming out of the openings of the plates: systems exist with rings or elastic strips, springs, stop notches, heat-retractable inserts, bolts, etc.

Although they have a certain effectiveness relative to the problem to be resolved, these systems have in common that they are relatively complex to manufacture and/or implement.

### BRIEF SUMMARY

The present invention therefore aims in particular to provide a plate-and-screw implant assembly that is very simple to manufacture and implement.

This aim of the invention is achieved with an implant assembly for bone fixation, and particularly for stabilizing the vertebral column, including a plate provided with at least two openings and at least two anchoring means that can be positioned in said openings and be attached to the bone portions to be stabilized, and blocking means for preventing said anchoring means from being accidentally released from said openings, remarkable in that blocking means include a shoulder arranged on each anchoring means on the one hand, and on the other hand an annular non-return lip which is arranged inside each opening, is integral with said plate, and can be deformed so as to be cleared by said shoulder during the positioning of the anchoring means, and then prevent the extraction of the anchoring means by forming an abutment against said shoulder.

Owing to this implant assembly according to the invention, the blocking means of the anchoring means (such as screws) in their openings are made in a single piece with the plate, and the blocking of those anchoring means in their openings is obtained due solely to the fixing of those anchoring means in the bone bodies, during which the shoulder of each anchoring means deforms and crosses the lip of the associated opening, then the lip is positioned abutting against said shoulder, preventing the anchoring means from being extracted.

According to other optional features of the implant according to the invention:
said shoulder and said non-return lip have profiles that are inclined in the direction of the introduction of each anchoring means into its opening, and straight in the direction of extraction of the anchoring means from its opening: this configuration makes it possible to facilitate the introduction of each anchoring means into its associated opening, and to achieve the desired anti-extraction effect simply;
said assembly comprises at least one piece reinforcing said plate forming seats for said anchoring means: the presence of a reinforcing piece makes it possible to increase the solidity of the plate in the cooperation zones with the anchoring means;
said reinforcing piece sandwiches said plate at least in the zones of said openings: this sandwich configuration makes it possible to obtain a very good distribution of the forces transmitted by the anchoring means to the plate;
said seats and the corresponding parts of said anchoring means are spherical: this configuration makes it possible to obtain ball joint-type connections between the anchoring means and the plate, making it possible to incline these anchoring means as needed as a function of the configuration of the vertebral bodies to be treated;
said plate is provided with four openings that can receive four anchoring means, two reinforcing means sandwiching said plate and each forming two seats for said anchoring means at two of said openings: this configuration may for example be suitable when one wishes to fix two anchoring means in each of the two vertebral bodies to be stabilized relative to one another;
the material forming said reinforcing piece is chosen from amongst the group made up of polymers, metal alloys, and ceramics;
said reinforcing piece is formed from a material harder than that forming said plate: this choice makes it possible to obtain the optimal strength of the connection of the anchoring means with the plate, while allowing the necessary flexibility for the non-return lips formed in the plate;
the material forming said plate is a polymer: such a material can make it possible to obtain an excellent compromise between a certain flexibility for the plate and the associated non-return lips, and very good strength relative to the forces transmitted to that plate by the vertebral bodies;
said anchoring means are screws each comprising a head and a thread, said shoulder being situated between this head and thread, and having a substantially annular shape, like said non-return lip.

### BRIEF DESCRIPTION OF THE DRAWINGS

Other features and advantages of the present invention will appear in light of the following description, and upon examining the appended figures, in which:

FIG. 1 diagrammatically shows an implant assembly according to the invention positioned on two vertebral bodies that must be stabilized relative to one another,

FIG. 2 is a perspective view of a plate of an implant assembly according to the invention,

FIG. 3 is a cutaway perspective view along line III of the plate of FIG. 2,

FIG. 4 shows a screw of this implant assembly, intended to cooperate with the plate of FIG. 2,

FIG. 5 shows an enlarged view of part V of FIG. 4,

FIG. 6 is a partial tear-away perspective view of the screw of FIGS. 4 and 5 cooperating with one of the openings of the plate of FIG. 2, during insertion of the screw, and

FIG. 7 is a view similar to that of FIG. 6, the screw being completely inserted into the plate.

In all of these figures, identical or similar references designate identical or similar members or sets of members.

### DETAILED DESCRIPTION

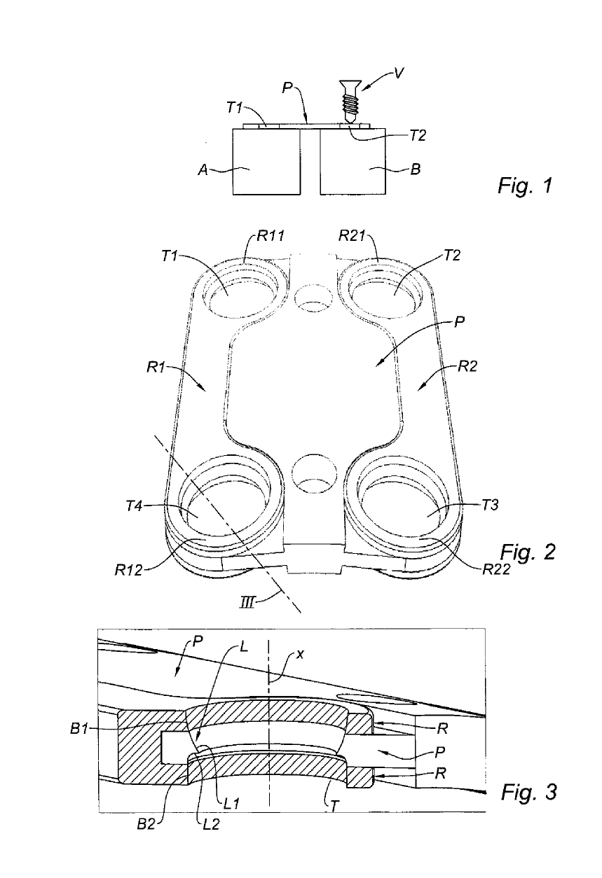

FIG. 1 diagrammatically illustrates the implant assembly according to the invention, during placement on two vertebral bodies A and B, which may for example be cervical vertebrae.

As shown, the implant assembly according to the invention comprises a plate P on the one hand provided with a plurality of openings T1, T2 positioned opposite the vertebrae A and B, and on the other hand a plurality of anchoring means V intended to be fixed in the vertebrae A and B while passing through the openings T1 and T2 of the plate P.

In the following, these anchoring means will be presented as screws, but this is in no way limiting: these anchoring means could in fact be nails, staples, cables, with or without shape memory, and with or without glue. The plate P makes it possible to immobilize the two vertebrae A and B completely or partially for the fusion or mutual stabilization thereof.

In the example shown in FIGS. 2 and 3, the plate P according to the invention has a rectangular general shape, pierced in all four corners thereof with four openings T1, T2, T3, T4.

The material forming the plate P can be made from polymer. As shown in FIGS. 2 and 3, the plate P is sandwiched by a first reinforcing piece R1, which therefore extends both above and below the plate P, and the ends R11 and R12 of which are configured to surround the openings T1 and T4, on both surfaces of the plate P.

A second reinforcing piece R2 is positioned symmetrically to the first reinforcing piece R1 and has the same structure as the piece R1, and the ends R21 and R22 thereof respectively surround the openings T2 and T3 of the plate P, on the two surfaces of that plate.

More specifically, as shown in FIG. 3, in each of the openings T1 to T4, the plate P is configured so as to define an annular non-return lip L, on the one hand comprising an edge L1 that is slightly inclined relative to the axis X of the corresponding opening T, and on the other hand an edge L2 that is substantially perpendicular to that axis X.

Also preferably (see FIG. 3), the edge B1 of the end of the reinforcing piece R that surrounds the opening T on the side of the lip L1 has a substantially spherical profile, the center of which is situated on the axis X, whereas the edge of the end of the piece R that is situated on the side of the edge L2 of the lip L has a cylindrical profile, i.e. substantially parallel to the axis X.

The material forming the reinforcing pieces R1, R2 can be chosen from amongst polymers, metal alloys, and ceramics, and is preferably harder than the material forming the plate P.

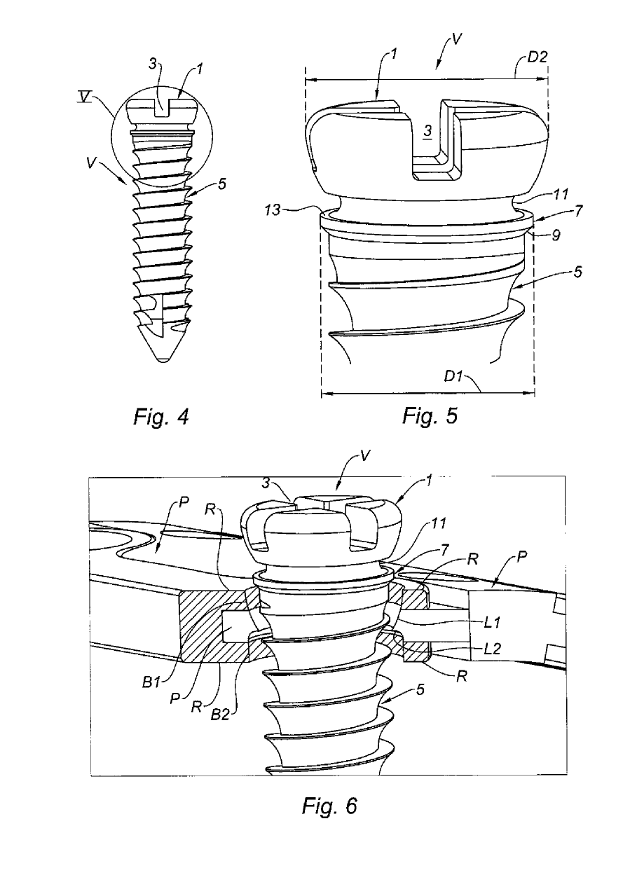

We will now refer to FIGS. 4 and 5, which show one of the four screws V intended to cooperate with the openings T1 to T4.

As shown in these two figures, this screw comprises a head 1 provided with a cavity 3 adapted to receive a tool with a complementary shape, as well as a threaded shaft 5, adapted to cooperate with the vertebral bodies A and B.

Between the head 1 and the threaded screw 5 of the screw V is an annular shoulder 7, the diameter D1 of which is slightly smaller than the smallest diameter of the openings T1 to T4 formed in the ends R11, R12, R21 and R22 of the reinforcing pieces R1 and R2, and larger than the smallest diameter of the lip L formed in the plate P in each of the openings T1 to T4.

The largest diameter D2 of the head 1 of the screw V is adapted to allow said screw head 1 to rest on the edge B1 of each opening T formed in the reinforcing pieces R1 and R2, so that the edge B1 constitutes the seat of that screw head.

More specifically, one can see that the shoulder 7 is connected to the threaded shaft 5 by a conical part 9, and to the screw head 1 by a hollow doughnut-shaped part 11.

The screw V can be formed from any material commonly used in the field of plate-and-screw implants, such as titanium-based metal alloys, for example.

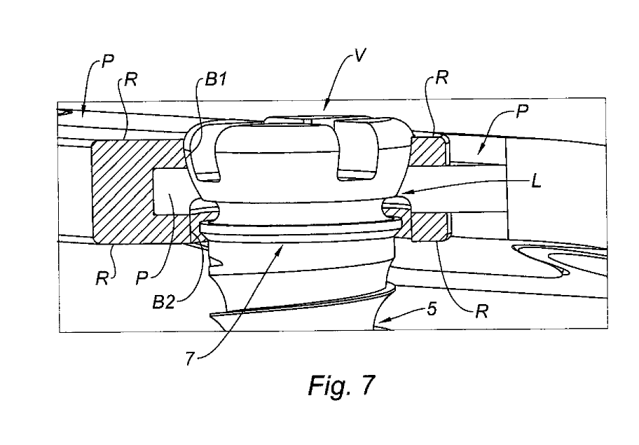

The usage mode and the advantages of the implant assembly just described will be particularly clear in light of the description of FIGS. 6 and 7.

When one wishes to fix this implant assembly on vertebral bodies A and B (see FIG. 1), the plate P is placed on those vertebral bodies, and the screws V are inserted into each of the openings T1 to T4, so that the threaded shafts 5 of those screws engage with the respective vertebral bodies.

These screws are therefore screwed using suitable tools and cooperating with the cavities 3 of the heads 1 of those screws, owing to which said screws rotate and are pushed into the associated vertebral bodies.

Due to the pushing in, there comes a moment where the conical part 9 of the shoulder 7 of each screw comes into contact with the slightly inclined edge L1 of the lip L of the concerned opening T.

When one continues to screw, the conical part 9 of the shoulder 7 deforms the lip L so as to push it radially back toward the outside of the opening T.

The cylindrical part of the shoulder 7 then moves along the slightly inclined part L1 of the lip L, until said cylindrical part has passed the connecting line between the slightly inclined edge L1 and the greatly inclined edge L2 of the lip L: at that stage, under the effect of the relative elasticity of the material forming the plate P, the lip L regains its initial shape and is housed inside the substantially doughnut-shaped part 11 separating the shoulder 7 from the screw head 1.

The greatly inclined edge L2 of the lip L is thus positioned abutting against the corresponding annular edge 13 of the shoulder 7.

One thus arrives at the situation of FIG. 7, in which the screw V is completely pushed to the inside of the associated opening T, the lip L then performing, due to its position in the doughnut-shaped part 11 abutting against the shoulder 7, a non-return function, making it possible to prevent the extraction of the screw V from the opening T.

Of course, by exerting substantial force, it remains possible to perform such an extraction, by going against the retaining force exerted by the lip L.

But it must be understood that such an extraction force requires a special tool, and that the aim of the invention is thus achieved, which is to prevent screws from leaving their openings spontaneously, when they are in use in a patient's body.

It will be noted that the cooperation of the head 1 of each screw V with the part forming the spherical seat B1 of each associated opening T, makes it possible to impart two degrees of freedom (ball joint-type connection) of that screw relative to the plate T, and thus to tilt said screws as needed as a function of the configuration of the vertebral bodies to be treated.

Advantageously, choosing a relatively flexible material for the plate P makes it possible to give the latter a certain deformability allowing a small relative movement of the vertebral bodies A and B, and using a relatively hard material for the reinforcing pieces R1 and R2 makes it possible to give sufficient strength to the seats of the screws V.

It will be noted that providing that the lips L are formed in the plate P itself constitutes a considerable simplification of the plate according to the invention, and that the relative flexibility of the material forming the plate P makes it possible to give those lips L the deformability necessary to allow the passage of each screw V and the subsequent blocking of the axial movement of that screw V in its associated opening T, owing to the repositioning of the associated lip L abutting against the shoulder 7 of that screw.

Of course, the present invention is in no way limited to the embodiment described and shown.

Thus the invention also covers a plate P without a reinforcing piece R1 and R2, i.e. formed in a rigid enough material to be able to constitute the seats of the heads of the screws V, and at the same time flexible enough to allow the deformation of the lips L associated with those openings.

The invention thus also extends to any plate with a shape different from that shown, and comprising a larger or smaller number of openings than those of that plate.

1. An implant assembly for bone fixation, and particularly for stabilizing a vertebral column, comprising:
a plate provided with at least two openings and at least two anchoring means that can be positioned in said openings and be attached to bone portions to be stabilized, and
blocking means for preventing said anchoring means from being accidentally released from said openings,
wherein blocking means include a shoulder arranged on each anchoring means and an annular non-return lip which is arranged inside each opening, is integral with said plate, and can be deformed so as to be cleared by said shoulder during positioning of the anchoring means, and then prevent extraction of the anchoring means by forming an abutment against said shoulder.

2. The assembly according to claim 1, wherein said shoulder and said non-return lip have profiles that are inclined in a direction of introduction of each anchoring means into its opening, and straight in a direction of extraction of the anchoring means from the opening.

3. The assembly according to claim 1, further comprising at least one piece reinforcing said plate forming seats for said anchoring means.

4. The assembly according to claim 3, wherein said reinforcing piece sandwiches said plate at least in zones of said openings.

5. The assembly according to claim 3, wherein said seats and corresponding parts of said anchoring means are spherical.

6. The assembly according to claim 5, wherein said plate is provided with four openings that can receive four anchoring means, two reinforcing means sandwiching said plate and each forming two seats for said anchoring means at two of said openings.

7. The assembly according to claim 3, wherein material forming said reinforcing piece comprises at least one of polymers, metal alloys, and ceramics.

8. The assembly according to claim 7, wherein said reinforcing piece is formed from a material harder than that forming said plate.

9. The assembly according to claim 1, wherein a material forming said plate is a polymer.

10. The assembly according to claim 1, wherein said anchoring means are screws each comprising a head and a thread, said shoulder being situated between this head and thread.
